(19) United States
(12) Patent Application Publication
(10) Pub. No.: US 2016/0090348 A1
(43) Pub. Date: Mar. 31, 2016

(54) PREPARATION OF C8-C24 ALKYL (METH)ACRYLATES

(71) Applicant: BASF SE, Ludwigshafen (DE)

(72) Inventors: Andrea MISSKE, Speyer (DE); Friederike FLEISCHHAKER, Ludwigshafen (DE); Christoph FLECKENSTEIN, Freigericht (DE); Martin KALLER, Mannehim (DE); Ulrik STENGEL, Birkenau (DE); Mathieu BLANCHOT, Lambsheim (DE); Ritesh NAIR, Heidelberg (DE)

(73) Assignee: BASF SE, Ludwigshafen (DE)

(21) Appl. No.: 14/854,141

(22) Filed: Sep. 15, 2015

Related U.S. Application Data
(60) Provisional application No. 62/057,266, filed on Sep. 30, 2014.

(30) Foreign Application Priority Data
Sep. 30, 2014 (DE) 102014219854.2

(51) Int. Cl. C07C 67/03; C07C 67/48

(52) U.S. Cl. CPC C07C 67/03; C07C 67/48

(57) ABSTRACT

The invention relates to a process for preparing a C₈-C₂₄ alkyl(meth)acrylate by transesterification of methyl(meth)acrylate with a C₈-C₂₄ alkanol, said process comprising the steps of:

(i) reacting methyl(meth)acrylate with the C₈-C₂₄ alkanol in the presence of a tin-comprising catalyst and a stabilizer in the presence of an entraining agent which forms an azeotrope with methanol,

(ii) continuously distilling off the azeotrope of entraining agent and methanol wherein steps (i) and (ii) are carried out simultaneously until the C₈-C₂₄ alkanol has been substantially completely reacted,

(iii) washing with an aqueous alkaline washing solution the C₈-C₂₄ alkyl (meth)acrylate-comprising product mixture obtained in steps (i) and (ii) to remove from the product mixture the tin-comprising catalyst and at least some of the stabilizer,

(iv) distilling off unconverted methyl(meth)acrylate and entraining agent from the product mixture,

(v) distilling off water from the product mixture

wherein a product having a by-product content of <0.5% by weight is obtained after step (v) and step (iv) may also be carried out before step (iii) and steps (iv) and (v) may also be carried out in one distillation step.

The invention relates to a process for preparing C₈-C₂₄ alkyl(meth)acrylates by transesterification of methyl (meth)acrylate with C₈-C₂₄ alkanols.

Polymers or copolymers prepared on the basis of branched or linear C₈-C₂₄ (meth)acrylates are of considerable economic importance in the form of polymer dispersions. They are used, for example, as adhesives, lubricants, oil field chemicals and paints and as textile, leather and papermaking assistants. (Meth)acrylic acid and (meth)acrylate are, respectively, umbrella terms for acrylic acid and methacrylic acid and for acrylate and methacrylate.

The preparation of (meth)acrylates alkylated with higher alkyl groups by acid-catalyzed esterification of (meth)acrylic acid with C₈-C₂4 alkanols is described in WO 2002/050014 A1 and WO 2002/050015 A1 for example.

WO 2009/106550 describes a process for preparing (meth)acrylates from C₁₀ alkanols by esterification of (meth)acrylic acid with an isomer mixture of C₁₀ alkanols comprising 2-propylheptanol as the main isomer in the presence of an acidic catalyst, a polymerization inhibitor and an entraining agent which forms an azeotrope with water wherein the esterification is carried out in a reactor equipped with a circulatory evaporator and the crude product obtained is purified by subsequent purifying distillation.

In the sole example, acrylic acid is esterified with 2-propylheptanol. Here, cyclohexane and 2-propylheptanol are initially charged and to this mixture are added the stabilizer hydroquinone monomethyl ether (MEHQ), hypophosphorous acid, copper(II) chloride solution and acrylic acid. The mixture is heated up under an air atmosphere, methanesulfonic acid is added and water is continuously removed under reflux. The cooled-down reaction solution is washed with sodium chloride solution and aqueous sodium hydroxide solution. The organic phase is removed and the entraining agent is removed under reduced pressure. 2-Propylheptyl acrylate is obtained in a yield of 97% with >95% purity and an APHA color index of 11. Once the purifying distillation has been carried out, 2-propylheptyl acrylate having a purity >99% and a lower APHA color index is obtained.

DE 10 2009 047 228 A1 discloses a process for preparing (meth)acrylates from C₁₇ alkanol mixtures by esterification of (meth)acrylic acid with a C₁₇ alkanol mixture in the presence of an acidic catalyst and a polymerization inhibitor and in the presence of an entraining agent which forms an azeotrope with water wherein the esterification is carried out in a reactor equipped with a circulatory evaporator and the azeotrope is distilled off and condensed. The C₁₇ alkanol mixture has a mean degree of branching of from 2.8 to 3.7. Useful acidic catalysts include mineral acids and sulfonic acids, such as sulfuric acid, phosphoric acid, alkylsulfonic acid, arylsulfonic acid and also acidic ion exchangers and zeolites.

In the example, acrylic acid is esterified with a C₁₇ alkanol mixture. This comprises initially charging cyclohexane, heptadecanol having a mean degree of branching of about 3.0, stabilizer (MEHQ), hypophosphorous acid and copper(II) chloride solution and then adding acrylic acid. The mixture is heated up under an air atmosphere, methanesulfonic acid is added and water is continuously removed under reflux. The cooled-down reaction solution is washed with sodium chloride solution and aqueous sodium hydroxide solution. The organic phase is removed and the entraining agent is removed under reduced pressure. Heptadecyl acrylate is obtained in a yield of 89% with >95% purity and an APHA color index of 9. A purifying distillation is not carried out since C₁₇ alkyl (meth)acrylates are of too high a molecular weight to be subjected to purifying distillation at acceptable cost.

Higher alkyl (meth)acrylates are also obtainable by catalytic transesterification of methyl (meth)acrylate with the appropriate long-chain alkanols. These reactions are carried out in the presence of a stabilizer (polymerization inhibitor).

DE 2 317 226 A1 discloses a process for preparing (meth)acrylic esters from a mixture of C₁₀-C₁₈ alkanols by transesterification of methyl (meth)acrylate in the presence of titanium alkoxide as catalyst and 2,6-di-tert-butylparacresol (TBC) as stabilizer. This process is carried out in the presence of activated carbon. Once the reaction has ended, water is added to hydrolyze the titanium alkoxide to titanium hydroxide/oxide which adsorbs onto the activated carbon. The solid is filtered off and the reaction product is subjected to a steam distillation.

WO 2009/080380 discloses a process for preparing methacrylates from C₆-C₂₂ alcohols by transesterification of methyl (meth)acrylate with the appropriate alcohols in the presence of titanium alkoxide as catalyst. In example 1, methyl methacrylate is reacted with 2-ethylhexanol in the presence of hydroquinone monomethyl ether (MEHQ) as stabilizer and tetraisopropyl titanate as catalyst. An azeotropic mixture of methanol/methyl methacrylate is distilled off here. Once unconverted methyl methacrylate has been distilled off, the 2-ethylhexyl methacrylate comprising catalyst is subjected to a purifying distillation under reduced pressure (about 30 mbar). This gives 2-ethylhexyl methacrylate in 99.4% purity.

The esterification of (meth)acrylic acid or the transesterification of (meth)acrylic esters with long-chain alkanols can generate not inconsiderable amounts of by-products by Michael addition. Such by-products are di- or oligo(alkyl(meth)acrylates) or oxyesters of the (meth)acrylic esters of either the reactant or product ester. These are high boilers with respect to the target product. Alkyl(meth)acrylates of long-chain alkanols can be removed from these by-products only by vacuum distillation and, when the reacted alkanols have more than a certain number of carbon atoms, only by distillation under greatly reduced pressure so that economical removal of the alkyl (meth)acrylates is not possible at all. Moreover, the catalyst employed and the stabilizer also need to be removed from the product. Provided that the boiling point of the target product is not too high, said target product is generally subjected to a final purifying distillation.

It is an object of the invention to provide a process for preparing C₈-C₂₄ alkyl (meth)acrylates which is simple to carry out and wherein the C₈-C₂₄ alkyl (meth)acrylates are obtained in high purity without a purifying distillation being carried out.

The object is achieved by a process for preparing a C₈-C₂4 alkyl (meth)acrylate by transesterification of methyl (meth)acrylate with a C₈-C₂₄ alkanol, said process comprising the steps of:
(i) reacting methyl (meth)acrylate with the C₈-C₂₄ alkanol in the presence of a tin-comprising catalyst and a stabilizer in the presence of an entraining agent which forms an azeotrope with methanol,
(ii) continuously distilling off the azeotrope of entraining agent and methanol wherein steps (i) and (ii) are carried out simultaneously until the C₈-C₂₄ alkanol has been substantially completely reacted,
(iii) washing with an aqueous alkaline washing solution the C₈-C₂₄ alkyl (meth)acrylate-comprising product mixture obtained in steps (i) and (ii) to remove from the product mixture the tin-comprising catalyst and at least some of the stabilizer,
(iv) distilling off unconverted methyl (meth)acrylate and entraining agent from the product mixture,
(v) distilling off water from the product mixture
   wherein a product having a by-product content of <0.5% by weight is obtained after step (v) and step (iv) may also be carried out before step (iii) and step (iv) and (v) can also be carried out in one distillation step.

It has been found that, surprisingly, transesterification of methyl (meth)acrylate with C₈-C₂₄ alkanols in the presence of a tin-comprising catalyst gives C₈-C₂₄ alkyl (meth)acrylates having a very low by-product content even without a purifying distillation. The washing step (iii) with an aqueous alkaline washing solution makes it possible to remove from the product mixture both the tin-comprising catalyst and at least some of the stabilizer in simple fashion by extraction and phase separation. Entraining agent, unconverted methyl (meth)acrylate and residual water may then be removed from the product mixture in simple fashion in a single distillation step.

The term by-products is understood to mean high-boiling compounds excluding the target product, in particular di- and oligo(meth)acrylic esters and oxyesters of the reactant and target monomer. The product obtained after step (v) may also comprise unconverted C₈-C₂₄ alkanols. These are not by-products. The C₈-C₂4 alkanol content of the product obtained in step (iv) is generally up to 0.5% by weight, preferably up to 0.3% by weight. The product obtained after step (v) may also comprise traces of entraining agent, methyl (meth)acrylate and water. These are likewise not by-products and may be present in the product obtained after step (v) in total amounts of up to 0.5% by weight, preferably 0.3% by weight.

The amount of all secondary components (including by-products, C₈-C₂₄ alkanols, entraining agent, methyl (meth)acrylate and water) is generally up to 1% by weight, preferably up to 0.5% by weight.

Preferred C₈-C₂₄ alkanols reacted in accordance with the inventive process are 2-propylheptanol and isomer mixtures of C₁₇ alkanols.

2-propylheptanol is generally employed as a C₁₀ alkanol mixture comprising 2-propylheptanol as the main isomer. The 2-propylheptanol content here is generally at least 50% by weight, preferably from 60% to 98% by weight, more preferably from 80% to 95% by weight and more particularly from 85% to 95% by weight, in each case based on the total weight of the C₁₀ alkanol mixture.

In addition to comprising 2-propylheptanol as the main isomer, the C₁₀ alkanol mixture generally also comprises at least one of the C₁₀ alcohols selected from the group consisting of 2-propyl-4-methylhexanol, 2-propyl-5-methylhexanol, 2-isopropylhexanol, 2-isopropyl-4-methylhexanol, 2-isopropyl-5-methylhexanol and 2-propyl-4,4-dimethylpentanol. 2-Propylheptanol may be prepared as described in DE 10 2007 001 540 A1.

Preferred C₁₇ alkanol mixtures have a C₁₇ alkanol content of at least 95% by weight, particularly preferably at least 98% by weight and more particularly at least 99% by weight based on the total weight of the C₁₇ alkanol mixture. Further preferred isomer mixtures of C₁₇ alkanols have a mean degree of branching (iso index) of from 2.8 to 3.7, particularly preferably from 2.9 to 3.6, more particularly from 3.05 to 3.4. The preparation of such C₁₇ alkanol mixtures is described in WO 2009/124979 A1.

The reaction of methyl (meth)acrylate with the C₈-C₂₄ alkanol is carried out in the presence of a catalyst comprising tin. Useful catalysts comprising tin are compounds comprising Sn(IV), for example dialkyltin dichloride, dialkyltin oxide, dialkyltin diacetate, bis(trialkyltin) oxide, bis(dibutylchlorotin) oxide, preference being given to dibutyltin dichloride, dimethyltin dichloride, dibutyltin oxide, very particular preference being given to dimethyltin dichloride. The chloride-containing catalysts may be employed together with alkoxides. Sodium methoxide is preferred here.

The reaction of methyl (meth)acrylate with the C₈-C₂₄ alkanol is carried out in the further presence of one or more stabilizers (polymerization inhibitors). Useful stabilizers may include, for example, N-oxides (nitroxyl or N-oxyl radicals, i.e., compounds bearing at least one >N—O group), for example 4-hydroxy-2,2,6,6-tetramethylpiperidine N-oxyl, 4-oxo-2,2,6,6-tetramethylpiperidine N-oxyl, 4-acetoxy-2,2,6,6-tetramethylpiperidine N-oxyl, 2,2,6,6-tetramethylpiperidine N-oxyl, bis(1-oxyl-2,2,6,6-tetramethylpiperidine-4-yl) sebacate, 4,4′,4″-tris(2,2,6,6-tetramethylpiperidine N-oxyl) phosphite or 3-oxo-2,2,5,5-tetramethylpyrrolidine N-oxyl; mono- or polyhydric phenols which may bear one or more alkyl groups, for example alkylphenols, for example o-, m- or p-cresol (methylphenol), 2-tert-butylphenol, 4-tert-butylphenol, 2,4-di-tert-butylphenol, 2-methyl-4-tert-butylphenol, 2-tert-butyl-4-methylphenol, 2,6-tert-butyl-4-methylphenol, 4-tert-butyl-2,6-dimethylphenol or 6-tert-butyl-2,4-dimethylphenol; quinones, for example hydroquinone, hydroquinone monomethyl ether, 2-methylhydroquinone or 2,5-di-tert-butylhydroquinone; hydroxyphenols, for example catechol (1,2-dihydroxybenzene) or benzoquinone; aminophenols, for example p-aminophenol; nitrosophenols, for example p-nitrosophenol; alkoxyphenols, for example 2-methoxyphenol (guaiacol, catechol monomethyl ether), 2-ethoxyphenol, 2-isopropoxyphenol, 4-methoxyphenol (hydroquinone monomethyl ether), mono- or di-tert-butyl-4-methoxyphenol; tocopherols, for example α-tocopherol and 2,3-dihydro-2,2-dimethyl-7-hydroxybenzofuran (2,2-dimethyl-7-hydroxycoumaran), aromatic amines, for example N,N-diphenylamine or N-nitrosodiphenylamine; phenylenediamines, for example N,N′-dialkyl-p-phenylenediamine wherein the alkyl radicals may be identical or different and each consist independently of from 1 to 4 carbon atoms and may be straight-chain or branched, for example N,N′-dimethyl-p-phenylenediamine or N,N′-diethyl-p-phenylenediamine, hydroxylamines, for example N,N-diethylhydroxylamine, imines, for example methyl ethyl imine or methylene violet, sulfonamides, for example N-methyl-4-toluenesulfonamide or N-tert-butyl-4-toluenesulfonamide, oximes such as aldoximes, ketoximes or amide oximes, for example diethyl ketoxime, methyl ethyl ketoxime or salicylaldoxime, phosphorous compounds, for example triphenylphosphine, triphenyl phosphite, triethyl phosphite, hypophosphorous acid or alkyl esters of the phosphorous acids; sulfur compounds, for example diphenyl sulfide or phenothiazine; metal salts, such as copper, manganese, cerium, nickel or chromium salts, for example chlorides, sulfates, salicylates, tosylates, acrylates or acetates, for example copper acetate, copper(II) chloride, copper salicylate, cerium(III) acetate or cerium(III) ethylhexanoate, or mixtures thereof.

Preference is given to hydroquinone, hydroquinone monomethyl ether, phenothiazine, 4-hydroxy-2,2,6,6-tetramethylpiperidine N-oxyl, 4-oxo-2,2,6,6-tetramethylpiperidine N-oxyl, 2-tert-butylphenol, 4-tert-butylphenol, 2,4-di-tert-butylphenol, 2-tert-butyl-4-methylphenol, 6-tert-butyl-2,4-dimethylphenol, 2,6-di-tert-butyl-4-methylphenol, 2-methyl-4-tert-butylphenol, hypophosphorous acid, copper acetate, copper(II) chloride, copper salicylate and cerium(III) acetate.

Polymerization inhibitors readily soluble in an aqueous alkaline washing solution, such as hydroquinone and hydroquinone monomethyl ether (MEHQ), are particularly preferred.

Advantageously, oxygen can additionally be used as a polymerization inhibitor.

To further support the stabilization, the reaction may be carried out in the presence of an oxygenous gas, preferably air or a mixture of air and nitrogen (lean air).

The transesterification reaction (steps (i) and (ii)) is generally carried out at a temperature of from 60° C. to 140° C., preferably from 70° C. to 110° C. An azeotrope of entraining agent and methanol is continuously distilled off here.

Useful entraining agents which form an azeotropic mixture with methanol are, in the first instance, methyl acrylate and methyl methacrylate themselves and also, as separate solvents, cyclohexane, methylcyclohexane, benzene, toluene, hexane, heptane and mixtures thereof. Preference is given to methyl acrylate, methyl methacrylate and mixtures of methyl (meth)acrylate with n-heptane and cyclohexane. The term entraining agent in this sense encompasses the reactant itself and any separate solvent additionally employed.

In one preferred embodiment, a separate solvent other than methyl (meth)acrylate is employed as entraining agent. Preferred solvents are n-heptane and cyclohexane. In this case, steps (iv) and (v) are carried out as a combined distillation step after step (iii).

In a further embodiment, no separate solvent is employed as entraining agent. In this case, the reactant methyl (meth)acrylate itself serves as entraining agent. In this case, step (iv) is carried out before step (iii). Step (v) is carried out after step (iv).

The entraining agent may subsequently be replenished in the reactor. To this end, one preferred embodiment comprises distilling off the azeotropic mixture of methanol and entraining agent via a suitable column, stirring said mixture with water in a mixing vessel and then transferring it into a phase separator, the methanol dissolving in water and the organic phase separating as top phase. The organic phase is preferably returned to the reaction mixture via the top of the column and thus recirculated save for small losses. It is alternatively possible to supply fresh entraining agent and work up the entraining agent-methanol mixture in a separate step or to dispense with replenishment of the entraining agent either completely or partially.

Methyl (meth)acrylate is generally employed in a stoichiometric excess. The excess of methyl (meth)acrylate per hydroxyl group to be esterified is preferably from 5 to 200 mol %, more preferably from 5 to 100 mol % and more particularly from 5 to 50 mol %. The solvent is generally employed in amounts of from 0% to 100% by weight, preferably from 0% to 50% by weight and more preferably from 0% to 20% by weight, based on the total amount.

The catalyst is employed in a concentration of from 0.1-10 mol % based on the amount of the C₈-C₂₄ alkanol employed, preferably in a concentration of from 0.1 to 5 mol %.

The transesterification may be carried out at atmospheric pressure but also at superatmospheric pressure or at reduced pressure. Said transesterification is generally carried out at from 300 to 1000 mbar, preferably at 800-1000 mbar (atmospheric pressure=1000 mbar).

The reaction time is generally from 1 h to 24 hours, preferably from 3 to 18 hours, more preferably from 6 to 12 h. The transesterification (steps (i) and (ii)) may be run as a continuous operation, for example in a stirred-tank cascade, or batchwise.

The reaction may be carried out in all reactors useful for a reaction of this type. Such reactors are known to those skilled in the art. The reaction is preferably carried out in a stirred-tank reactor.

The batch may be commixed using any desired processes such as stirring means for example; commixing may also be achieved by injection of a gas, preferably an oxygen-containing gas.

The methanol generated is removed either continuously or stepwise in a manner known per se by azeotropic distillation in the presence of an entraining agent. In addition, methanol may also be removed by stripping with a gas.

In one preferred embodiment, methanol is removed from the azeotrope of entraining agent and methanol distilled off in step (ii) by washing with water and the entraining agent is recycled to the reaction vessel.

Steps (i) and (ii) are carried out until substantially all of the C₈-C₂₄ alkanol has reacted. This is the case when 98%, preferably 99% and more preferably 99.5% of the C₈-C₂₄ alkanol has reacted.

Steps (iii) and (iv) follow and may also be carried out in reverse order.

One embodiment comprises initially carrying out at least one washing step (iii) wherein the product mixture comprising C₈-C₂₄ alkyl (meth)acrylate is contacted with an aqueous alkaline washing solution. Useful aqueous alkaline washing solutions are aqueous alkali metal hydroxide solution, preferably aqueous sodium or potassium hydroxide solution, more preferably aqueous sodium hydroxide solution. The concentration thereof is generally from 1% to 20% by weight and preferably from 5% to 15% by weight. The alkaline washing step may be followed by a neutral washing step with water and an acidic washing step with very dilute mineral acid, for example phosphoric acid, generally having a mineral acid content of from 0.1% to 1% by weight.

The wash may be carried out, for example, in a stirred vessel or in another conventional apparatus, for example in a column or mixer-settler apparatus.

In process engineering terms, any wash in the process according to the invention may be carried out using all extraction and washing processes and apparatuses known per se, for example those described in Ullmann's Encyclopedia of Industrial Chemistry, 6th ed, 1999 Electronic Release, chapter “Liquid-Liquid Extraction-Apparatus”. For example, these may be single-stage or multistage, preferably single-stage, extractions and also extractions in cocurrent or countercurrent mode.

In one preferred embodiment, unconverted methyl (meth)acrylate, separate solvent and traces of water are subsequently distilled off from the product mixture in distillation steps (iv) and (v).

This distillation is generally carried out at a temperature of from 40° C. to 100° C., preferably from 60° C. to 80° C., and a variable pressure of from 10 to 700 mbar. In addition, these components may also be removed by stripping with a gas, preferably an oxygenous gas.

The distillative removal is carried out, for example, in a stirred tank with jacket heating and/or internal heating coils under reduced pressure.

It will be appreciated that the distillation may also be carried out in a falling-film or thin-film evaporator. To this end, the reaction mixture is passed through the apparatus, preferably two or more times in a circuit, under reduced pressure, for example at from 20 to 700 mbar, preferably from 30 to 500 mbar and more preferably from 50 to 150 mbar, and at a temperature of from 40° C. to 80° C.

An inert gas, preferably an oxygenous gas and more preferably air or a mixture of air and nitrogen (lean air), may advantageously be introduced into the distillation apparatus, for example from 0.1 to 1, preferably from 0.2 to 0.8 and more preferably from 0.3 to 0.7 m³/m³h, based on the volume of the reaction mixture.

Once steps (iii), (iv) and (v) have been carried out, there remains a product in the form of a bottoms product which is obtained in the purity described hereinabove.

The invention is more particularly described using the examples which follow.

### EXAMPLES

### Example 1

A 4 L flange reactor equipped with a column (Sulzer CY packing), cooler, liquid divider, cross-beam stirrer, air introduction means and an apparatus for washing the organic phase having a downstream phase separator and automatic recycling of the organic phase is initially charged with heptane (180 g), methyl acrylate (806 g), MeHQ (6.3 g), dimethyltin dichloride (44.89 g), 30% strength sodium methoxide solution, methanol (15.69 g) and C₁7 alcohol (1500 g) and heated up to a column-bottom temperature of 84° C. with air introduction and stirring. Once the mixture has started to boil, the reflux ratio is set to 6:4. The amount of wash water is continuously adjusted according to the amount of distillate present. The column-bottom temperature rises to 109° C. over the course of the reaction. Bottoms are sampled at regular intervals to monitor the course of the reaction.

Once the reaction has ended, the mixture is cooled down to 50° C. and the reaction mixture is admixed with 200 g of heptane. The mixture is washed 3× with 10% strength sodium hydroxide, then with water and with 0.35% strength aqueous phosphoric acid. The aqueous phases are removed and discarded in each case. The heptane, MMA and residual water are then distilled off at 80° C. under full vacuum. The product is obtained in 1746 g (95.7%) yield and 99.6% purity. The color index is 145 Hazen and the MEHQ content is 60 ppm.

### Example 2

A 4 L flange reactor equipped with a column (Sulzer CY packing), cooler, liquid divider, cross-beam stirrer, air introduction means and an apparatus for washing the organic phase having a downstream phase separator and automatic recycling of the organic phase is initially charged with heptane (180 g), methyl methacrylate (937 g), MeHQ (0.91 g), dimethyltin dichloride (44.89 g), 30% strength sodium methoxide solution, methanol (15.69 g) and C₁₇ alcohol (1500 g) and heated up to a column-bottom temperature of 96° C. with air introduction and stirring. Once the mixture has started to boil, the reflux ratio is set to 6:4. The amount of wash water is continuously adjusted according to the amount of distillate present. The column-bottom temperature rises to 109° C. over the course of the reaction. Bottoms are sampled at regular intervals to monitor the course of the reaction.

Once the reaction has ended, the mixture is cooled down to 50° C. and the reaction mixture is admixed with 200 g of heptane. The mixture is washed 1× with 10% strength sodium hydroxide, then with water and with 0.35% strength aqueous phosphoric acid. The aqueous phases are removed and discarded in each case. The heptane and residual water are then distilled off at 80° C. under full vacuum. The product is obtained in 1811 g (94%) yield and 98.5% purity. The color index is 17 Hazen and the MEHQ content is 90 ppm.

The quantitative analysis of the secondary components in the products obtained is carried out using GC, HPLC (stabilizer) and by titrimetry ((meth)acrylic acid, water). The results are summarized in Table 1.

### Example 3

A 4 L flange reactor equipped with a column (Sulzer CY packing), cooler, liquid divider, cross-beam stirrer, air introduction means and an apparatus for washing the organic phase having a downstream phase separator and automatic recycling of the organic phase is initially charged with cyclohexane (300 g), methyl acrylate (1391 g), MeHQ (0.6 g), dimethyltin dichloride 70% in methanol (31.4 g), 30% strength sodium methoxide solution in methanol (18 g) and 2-propylheptanol (1583 g) and heated up to a column-bottom temperature of 86° C. with air introduction and stirring. Once the mixture has started to boil, the reflux ratio is set to 2:4.1. The amount of wash water is continuously adjusted according to the amount of distillate present. The column-bottom temperature rises to 114° C. over the course of the reaction. Bottoms are sampled at regular intervals to monitor the course of the reaction.

Once the reaction has ended, the mixture is cooled down to 45° C. The mixture is washed once with 10% strength sodium hydroxide, then with water and 1% strength aqueous phosphoric acid. In each case the aqueous phases are removed and discarded. 100 mg of MeHQ are then admixed with the mixture and the cyclohexane and residual water are distilled off at 75° C. under full vacuum. The product is obtained in 2114 g (99.6%) yield and 99.4% purity (GC area %). Detailed analytical results are shown in Table 1.

### Example 4

A 4 L flange reactor equipped with a column (Sulzer CY packing), cooler, liquid divider, anchor stirrer and an air introduction means is initially charged with methyl acrylate (1772 g), MeHQ (0.66 g) and 2-propylheptanol (1583 g) and heated up to a column-bottom temperature of 39° C. with air introduction and stirring. Dimethyltin dichloride (1.54 g) and a 30% strength solution of sodium methoxide in methanol (0.54 g) are metered into the mixture and heated up further to a column-bottom temperature of 89° C. Once the mixture has started to boil, the reflux ratio is set to 5:4.1 and later 5:2. The column-bottom temperature rises to 105° C. over the course of the reaction. Bottoms and distillate are sampled at regular intervals to monitor the course of the reaction. The pressure is reduced to a final pressure of 480 mbar to complete the reaction. An end temperature of 100° C. is reached. Residual methyl acrylate is then distilled off under full vacuum.

The experiment is cooled down to 40° C. and washed initially with 750 g of 10% strength NaOH, then with water and finally with 0.35% strength phosphoric acid, the phases being separated in each case. The residual water is distilled off at 80° C. under full vacuum. The product is obtained in 1996 g (94% yield) and 98.6% purity (GC area %) and has a color index of 8 Hazen.

### Example 5

The plant described in Example 1 is initially charged with an alcohol mixture of C₁₆/C₁₈ alcohol (Hydrenol D, 2068 g), methyl methacrylate (1051 g) and cyclohexane (374 g), and dimethyltin dichloride (1.80 g), a 30% strength sodium methoxide solution in methanol (1.44 g) and MEHQ (10.5 g) are admixed therewith and the resulting mixture is heated to boiling point (about 82° C.) at atmospheric pressure with air introduction. Once the mixture has started to boil, the reflux ratio is set to 2:4.2. During the reaction, the amount of wash water necessary to remove methanol from the distillate is continuously adjusted according to the quantity of distillate obtained. The mass lost from the reactor due to methanol removal is compensated on an hourly basis by addition of methyl methacrylate. The reaction mixture temperature rises to 117° C. over the course of the transesterification. Samples are taken from the reaction mixture at regular intervals and analyzed by gas chromatography in order to monitor the progress of the reaction.

Once the reaction has ended (about 11 h), all low-boiling components (cyclohexane and excess methyl methacrylate) are removed under reduced pressure (60° C., 10 mbar) and the crude product, once cooled, is admixed with cyclohexane (374 g). The mixture is washed successively with 10% strength aqueous sodium hydroxide solution (599 g), water (524 g) and 0.35% strength aqueous phosphoric acid (262 g), once in each case. In each case, the aqueous phases are removed and discarded. The washed crude product is stabilized with MEHQ (0.16 g) and cyclohexane and also traces of water are removed under reduced pressure (64° C., 11 mbar). The product is obtained in 98% yield and 98.4% purity (GC area%). The color index (Hazen) is 30, the MEHQ content (HPLC) is 180 ppm and the tin content is <3 ppm (method detection limit).

### Example 6

The plant described in Example 1 is initially charged with a C₁₈-C₂₂ alcohol mixture (Stenol 1822

SR, 2068 g), methyl acrylate (904 g) and cyclohexane (394 g), and dimethyltin dichloride (1.80 g), a 30% strength sodium methoxide solution in methanol (1.44 g) and MEHQ (10.5 g) are admixed therewith and the resulting mixture is heated to boiling point (about 99° C.) at atmospheric pressure with air introduction. Once the mixture has started to boil, the reflux ratio is set to full distillate take off. During the reaction, the amount of wash water necessary to remove methanol from the distillate is continuously adjusted according to the quantity of distillate obtained. The mass lost from the reactor due to methanol removal is compensated on an hourly basis by addition of methyl acrylate (256 g in total). The reaction mixture temperature rises to 104° C. over the course of the transesterification. The progress of the reaction is monitored by sampling the reaction mixture at regular intervals and analyzing the samples by gas chromatography.

Once the reaction has ended (about 12 h), all low-boiling components (cyclohexane and excess methyl acrylate) are removed under reduced pressure (100° C., 35 mbar) and the crude product, once cooled, is admixed with cyclohexane (374 g). The mixture is washed successively with 10% strength aqueous sodium hydroxide solution (599 g), water (524 g) and 0.35% strength aqueous phosphoric acid (262 g), once in each case. The aqueous phases are removed and discarded in each case. The washed crude product is stabilized with MEHQ (0.16 g) and cyclohexane and also traces of water are removed under reduced pressure (65° C., 15 mbar). The product is obtained in 96.6% yield and 99.7% purity (GC area %). The color index (Hazen) is 9 and the tin content is <3 ppm (method detection limit).

### Comparative Example 1a

A 2 L four-necked flask equipped with a thermometer, stirrer, water trap and air introduction means is initially charged with cyclohexane (360 g), 2-propylheptanol (634 g), MeHQ (246 mg), 50% strength hypophosphorous acid (616 mg) and Cu(II) chloride solution (20% strength, 0.54 mL). Acrylic acid (318 g, stabilized with 200 ppm MeHQ) is subsequently metered into the mixture. p-Toluenesulfonic acid monohydrate (22.8 g) is added. The mixture is heated up with air introduction and with stirring at a bath temperature of from 105° C. to 110° C. Water passes over at an internal temperature of 85-99° C. After 5 h, the reaction mixture is cooled down and admixed with 200 mL of cyclohexane. The reaction mixture is washed with 6.5% strength aqueous NaCl solution, with aqueous NaOH solution (266 mL of water and 80 mL of 25% strength NaOH) and once more with 6.5% strength NaCl solution. Following phase separation, the organic phase is admixed with 160 mg of MEHQ and concentrated under reduced pressure. The product is obtained in 840 g (99%) yield and 96.8% purity.

### Comparative Example 1b

The crude product obtained in the comparative example was subjected to distillative purification in a laboratory falling-film evaporator (area 0.046 m²). The pressure was kept constant at 3 mbar and the rotation frequency was 500 rpm. The jacket temperature was set to a temperature of from 113° C. to 114° C. The feed rate was from 0.6 to 1 kg/h. The product was obtained in 94% yield and 98.8% purity.

TABLE 1

| | | | Comparative | Comparative |
|---|---|---|---|---|
| | Example 3 | Example 4 | example 1a | example 1b |
| | Process | | | |
| | Trans- | Trans- | | |
| | esterification | esterification | | Esterification |
| | with | without | | and |
| | solvent | solvent | Esterification | distillation |
| | Analytical results | | | |
| | % by weight | % by weight | % by weight | % by weight |
| Purity | 99.42 | 97.08 | 96.75 | 98.82 |
| Sum of | 0.54 | 2.83 | 3.19 | 1.14 |
| impurities | | | | |
| Sum of | 0.23 | 0.24 | 2.3 | 0.58 |
| by-products | | | | |
| Water | 0.02 | 0.05 | 0.02 | 0.01 |
| Acrylic acid | n.d. | 0.002 | 0.01 | 0 |
| MEHQ | 0.01 | 0.04 | 0.02 | 0.03 |
| Cyclohexane | 0.08 | 0 | 0.48 | 0.11 |
| Sum of | 0.23 | 2.59 | 0.41 | 0.45 |
| propylheptanols | | | | |
| Sum of | 0.16 | 0.17 | 0.24 | 0.23 |
| unknown | | | | |
| peaks | | | | |
| Isopropylheptyl | 0 | 0 | 0.15 | 0 |
| oxyester | | | | |
| Sum of | 0 | 0 | 0.55 | 0.14 |
| di(propylheptyl | | | | |
| acrylates) | | | | |
| 2- | 0.03 | 0.023 | 0.17 | 0.2 |
| Propylheptyl | | | | |
| acetate | | | | |
| 2- | 0.01 | 0.01 | 1.18 | 0 |
| Propylheptyl | | | | |
| oxyester | | | | |
| 2- | 0.03 | 0.03 | 0.02 | 0.02 |
| Propylheptyl | | | | |
| propionate | | | | |

1. A process for preparing a C₈-C₂₄ alkyl (meth)acrylate by transesterification of methyl (meth)acrylate with a C₈-C₂₄ alkanol, said process comprising the steps of:
(i) reacting methyl (meth)acrylate with the C₈-C₂₄ alkanol i the presence of a tin-comprising catalyst and a stabilizer in the presence of an entraining agent which forms an azeotrope with methanol,
(ii) continuously distilling off the azeotrope of entraining agent and methanol wherein steps (i) and (ii) are carried out simultaneously until the C₈-C₂₄ alkanol has been substantially completely reacted,
(iii) washing with an aqueous alkaline washing solution the C₈-C₂₄ alkyl (meth)acrylate-comprising product mixture obtained in steps (i) and (ii) to remove from the product mixture the tin-comprising catalyst and at least some of the stabilizer,
(iv) distilling off unconverted methyl (meth)acrylate and entraining agent from the product mixture,
(v) distilling off water from the product mixture
wherein a product having a by-product content of <0.5% by weight is obtained after step (v) and step (iv) may also be carried out before step (iii) and steps (iv) and (v) may also be carried out in one distillation step.

2. The process according to claim 1 wherein the entraining agent is a separate solvent other than methyl (meth)acrylate and steps (iv) and (v) are carried out in one distillation step.

3. The process according to claim 2 wherein the solvent is selected from the group consisting of n-heptane and cyclohexane.

4. The process according to claim 1 wherein the entraining agent is methyl (meth)acrylate and step (iv) is carried out before step (iii).

5. The process according to claim 1 wherein the C₈-C₂₄ alkanol is a branched C₈-C₂₄ alkanol.

6. The process according to claim 1 wherein the C₈-C₂₄ alkanol is a linear C₈-C₂₄ alkanol.

7. The process according to claim 6 wherein the C₈-C₂₄ alkanol is a linear C₈-C₂₄ alkanol mixture.

8. The process according to claim 5 wherein the C₈-C₂₄ alkanol is a C₁₀ alkanol mixture comprising 2-propylheptanol.

9. The process according to claim 1 wherein the C₈-C₂₄ alkanol is a C₁₇ alkanol mixture.

10. The process according to claim 1 wherein the catalyst comprises dimethyltin dichloride.

11. The process according to claim 1 wherein the stabilizer is methylhydroquinone.

12. The process according to claim 1 wherein the aqueous alkaline washing solution is aqueous sodium hydroxide solution.

13. The process according to claim 1 wherein methanol is removed from the azeotrope of entraining agent and methanol distilled off in step (ii) by washing with water and the solvent is recycled to the reaction vessel.
